(19) United States
(12) Patent Application Publication
(10) Pub. No.: US 2005/0142223 A1
(43) Pub. Date: Jun. 30, 2005

(54) Pharmacological activity and manufacturing method of the production produced by 16 kinds of plant compound to improve memory

(76) Inventors: Jiao Gong, Arcadia, CA (US); Wei Jiang, Arcadia, CA (US)

Correspondence Address: JIAO GONG, 411 S. 3RD AVE #B, ARCADIA, CA, 91006, US

(21) Appl. No.: 10/745,794

(22) Filed: Dec. 29, 2003

(51) Int. Cl. A61K 35/78

(52) U.S. Cl. 424/728; 424/746; 424/757; 424/773; 424/777

(57) ABSTRACT

The medicine for delaying senescence and improving intellect are classified into eight kinds and five kinds respectively, which have brought inconvenience of taking to the patients who are eager to improve memory and delay senescence. The production produced by sixteen kinds of plant compound to improve memory applies itself to synthesize the eight kinds of medicine for delaying senescence and five kinds of medicine for improving intellect, selects sixteen kinds of plants into a compound, separate and purify natural production with WLD resin absorbing and dewatering technic under careful and strict condition. The production produced by sixteen kinds of plant compound to improve memory could improve memory hurt impediment by scopolamine distinctly It could decrease LPO in brain and liver, improve the activity of SOD in sedation, hypnosis and memory. The curative results to patiets with forgettery and insomnia in clinic validate the veracity of of pharmic activity. The pharmic activity of the compound production has presented a wide application future for its' medical use

### BACKGROUND OF THE INVENTION

The pharmic activity and manufacturing method of the production produced by sixteen kinds of plant compound to improve memory is a kind of combination patent of American patents which is combined by the methods of natural production pharmic activity and separation, purification natural production. The experiment of pharmic activity confirms that the sixteen kinds of plant compound have effect of improving memory, sedation, soothing the nerves and hypnosis, and the production are medicine to anti caducity and a supplyment to health which are safe and can improve intellect.

The Court of Patent and Cassation of America deem that the pharmic activity knowledge of any compound distinctively benefit the public. When the medicinal industry has known the pharmic activity of the compound, it is naturally faster and more easily to conquer and lighten disease. To open the pharmic activity of compound as more as possible, it is most important to offer encouragement for investigator. The court hereby draws the conclusion to meet the practicality by afforming the invention of the pharmic activity of compound. The compound specilied by the Court of Patent and Cassation of America doesn't be specified as single chemical structure compound. It should be identified as compound when multi-structure compounds were mixed together. So the pharmic activity and manufacturing method of the production produced by sixteen kinds of plant compound to improve memory should meet the requirements of Exclusive Law of America.

Senescence aging is a necessary process of living activity of biologic organization and is a natural rule which no one can dispute. However, the speed of Senescence aging can be delayed and the life can be extended under certain terms. It is of important meaning for improving senile biology, senile medicine and life quality of Americans to search the antiaging measurements and the antiaging drugs and antisenility drugs. The antiaging drugs and antisenility drugs are defined as these medicines to delay senescence aging, extend life and improve living energy. It is classified into eight sorts: antioxidant, antioxidant enzyme, brain antiaging galenical, immunity function regulator, monoamine oxidase depressor, microelement galenical, micro zoology galenical, Hyperpolyalkenacid, plant galenical. The production produced by sixteen kinds of plant compound to improve memory synthesize the eight kinds of antiaging drugs, complement each other and cooperate together to treat synthetically and delay senescence aging.

Memory impediment is common clinical symptom in elders, and is also an important symbol of aging. The elders are commonly nervous for oblivion, especially both the Senile dementia and Alzheimer's disease proper to the elders have memory impediment. The clinical symptoms of memory impediment bring much inconvenience to life. Therefore, it is of important clinical meaning to study medicine to improve intellect. The representative medicine to improve intellect in American market is ramification of pyrrolidone, the pyrrolidone is a medicine to metabolize and improve the brain energy, it increase the micro circulation function of nerve center system and improve memory and study ability. Such medicines include Pizacetam, Oxiracetam, Aniracetam. The natural plant element in the production produced by sixteen kinds of plant compound to improve memory are convenient to take at any time, it will add an excellent work to the drugs to improve intellect when after being used in American medical field.

### BRIEF SUMMARY OF THE INVENTION

To search and develop drugs to delay aging and improve intellect is the hottest topic in American medical field. The scientists are working untiringly day and night to improve human's memory and delay aging. The drugs to delay aging in American are classified into eight sorts: antioxidant, antioxidant enzyme, brain antiaging galenical, immunity function regulator, monoamine oxidase depressor, microelement galenical, micro zoology galenical, Hyperpolyalkenacid, plant galenical. The drugs to improve intellect in American are classified into five sorts: brain cells metabolizing and activation galenical, brain blood circulation accelerating galenical, calcium hydronium antagonism, improving cholin energy drugs and plant galenical. People are eager to improve memory and delay senescence aging, facing so many kinds of drugs with different characters they really don't know which one to select. The production produced by sixteen kinds of plant compound to improve memory take the assembly of drugs to delay aging and improve intellect as it's developing direction and collocate elaborately the sixteen kinds of plant compound by selecting though hundred times of experiments in order to obtain the effect of delaying aging and improving intellect synthetically and concurrently in all respects. The production is easily to be taken with multifunctions and have the remarkable effect by taking daily.

The production produced by sixteen kinds of plant compound to improve memory adopt the technology of WLD resin adsorption and dewatering, separate and purify the natural production, detect ration thumbprint atlas elements with high effective liquid item chromatogram detector and guarantee the quality stability of the production. The pharmic activity experiment indicates that the compound production could improve the memory of little white rats, decrease times of wrong reaction of the rats in Y type labyrinth, and could decrease the degree of memory hurt after the rats being injected Hydrogen bromic acid scopolamine, decrease wrong reaction times of the hurting rats in Y type labyrinth. The compound production could improve the big white rat's ability of studying memory, decrease times of wrong actions of the elder white mice after learning how to memorize. The pharmic activity experiment indicates that the compound production not only could improve studying memory of normal animals, but also could improve the hurting memory of the little white rats hurt by scopolainine, improve studying memory and memory hurt. The compound production could increase the activity of SOD in the blood of elder white rats, decrease the form of lipid peroxide in the brain and liver of the elder white rats, improve distinctively the activity of SOD in the blood, and present certain relation of quantity and effect. The compound production has better effect of sedation and hypnosis, and cooperate hypnosis with pentobarbital sodium together. (please refer to drawing 3).

The clinical observation of the production produced by sixteen kinds of plant compound to improve memory of 100 patients memory indicates that distinct effect of the compound production accounts for 45%, the total effective rate is 89%, the compound production has distinct effect to insomnia, the effect of taking the production for four weeks is much better than that for two weeks. For forgettery patient, the effect of taking the production for four weeks is better than that for two weeks. The electroencephalogram are both normal whether checking the electroencephalogram before the treatment or after it. After taking the compound production, all of the blood routine, stale routine and liver function doesn't change before or after treatment. There appear no poisonous and side reactions at the end of treatment. The LPO before treatment is 18.56 nmol/ml which is higher than normal level, and the LPO decrease to 10.47% nmol/ml which is in normal area after taking the compound production, the compound production has distinct effect in decreasing the LPO in blood. (Please refer to drawing four)

### BRIEF DESCRIPTION OF THE SEVERAL VIEWS OF THE DRAWING

### Table of Contents

Table 1 The compose and source of material of the production produced by sixteen kinds of plant compound to improve memory

Table 2 Manufacturing method and technics flow of the production produced by sixteen kinds of plant compound to improve memory

Table 3 The phartic activity of the production produced by sixteen kinds of plant compound to improve memory

Table 4 The clinical observation of the production produced by sixteen kinds of plant compound to improve memory

TABLE 1
The compose and source of material of the production produced by
sixteen kinds of plant compound to improve memory

| | | |
|---|---|---|
| 1. | Rhizoma Acori Tatarinowii | 8.58% |
| | Araceae | |
| | Acorus Tatarinowii Schott's rhizoma | |
| 2. | Radix Rehmanniae | 11.35% |
| | Scrophulriaceae | |
| | Rahmannia Glutinosa Libosch's earthnut | |
| 3. | Radix Ginseng | 5.67% |
| | Araliaceae | |
| | Panax Ginseng C.A. Mey's root | |
| 4. | Radix Polygoni Multiflori | 5.67% |
| | Polygonaceae | |
| | Polygonum Multiflorum Thunb's earthnut | |
| 5. | Cortex Eucommiae | 5.67% |
| | Eucommiaceae | |
| | Eucommia Ulmoides Oliv's bark | |
| 6. | Fructus Corni | 5.67% |
| | Cornaceae | |
| | Cornus Offcinalis Sieb.etzuce's fruit | |
| 7. | Fructus Schisandrae | 5.67% |
| | Magnoliaceae | |
| | Schisandra Chineusis(Turea)Baill's fruit | |
| 8. | Radix Polygalae | 2.83% |
| | Polygalaceae | |
| | Polygala Tenuifolia Willd's root | |
| 9. | Poria | 5.67% |
| | Poria Cocos(Schw.)Wolf's dry fungus pit | |
| 10. | Semen Placycladi | 5.67% |
| | Platycladus Orientalis(L.)Franco's seed | |
| 11. | Radix Saposhnikovia | 5.67% |
| | Umbelliferae | |
| | Saposhnikovia Divaricata(Turcz.) | |
| | Schischk's root | |
| 12. | Balbus Fritillariae Cirrhosae | 5.67% |
| | Liliaceae | |
| | Fritillaria Cirrhosa D.Don's bulb | |
| 13. | Radix Ophiopogoins | 11.35% |
| | Liliaceae | |
| | Ophiopogon Japonicus(Thumb.) | |
| | Ker-Gawl's earthnut | |
| 14. | Radix Salviae Miltiorrhizae | 5.67% |
| | Asclepiadaceae | |
| | Salvia Miltiorrhiza Bge's rhizoma | |
| 15. | Eadix Notoginseng | 3.54% |
| | Araliaceae | |
| | Panax Notoginseng (Burk)F.H.chen's earthnut | |
| 16. | Radix Glycyrrhizae | 5.67% |
| | Leguminosae | |
| | Glycyrrhiza Uralensis Fisch's rhizoma | |
| | In total | 100% |

TABLE 2
Manufacturing method and technics flow of the production produced by
sixteen kinds of plant compound to improve memory

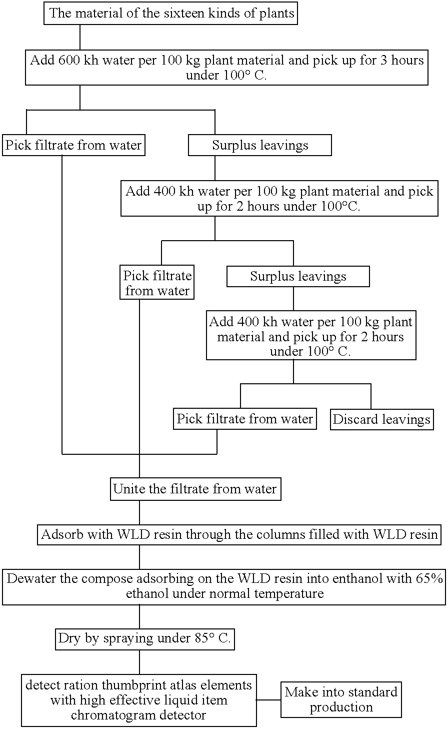

Table 3 The Phartic Activity of the Production Produced by Sixteen Kinds of Plant Compound to Improve Memory

The production produced by sixteen kinds of plant compound to improve memory (in brief compound production) have effects of improving memory, sedation, soothing the nerves and hypnosis, by medicine effective study review, the compound production have such pharmic activity as follows.

The Production Produced by Sixteen Kinds of Plant Compound to Improve Memory

Sulfer barbital acid, Four second oxygen radix propane, Flukd, Hydrogen bromic acid scopolamine, Adriok, SOD detecting reagent box, pentobarbital sodium, Union.

Method and Result:

1. The Influence the Compound Production Acts on the Little White Rats

Get little white rats weighing 1 8˜20 g and group them by random, ten rats each group and give them compound production. Wait thirty minutes after the last medicine, put the little whit rats in the Y type labyrinth according to the methods in the literature which have been modified a little and train them. At first put the little whit rats in the Y type labyrinth for five minutes to accormodate, then connect 36V voltage, the little whit rats who response to nine series stimulating out of ten are considered to be up to grade. After 24 hours put the little whit rats in the Y type labyrinth again, stimulate them with the same voltage for ten times and record the percentage of wrong reactions, the results are as follows in table 1:

TABLE 1
the influence the compound production acts on the little white rats

| | | | | Times of wrong |
|---|---|---|---|---|
| Medicine | Dose (ml/kg) | Taking time | n | reactions (\overline{X} ± SD) |
| NS | Same capacity | Bid × 7 | 10 | 0.425 ± 0.114 |
| Compound | 4 | Bid × 7 | 10 | 0.293 ± 0.126** |
| production | | | | |
| Compound | 8 | Bid × 7 | 10 | 0.211 ± 0.093*** |
| production | | | | |

**P < 0.05
***P < 0.01 compared to NS

2. The influence the Compound Production Acts on the Little White Rats which have Memory Hurt by Scopolamine

Get little white rats weighing 18˜20 g and group them by random, ten rats each group and give them compound production, fill stomach with same capacity of sanitation brine for memory hurt pathology model. Wait sixty minutes after the last medicine, expect blank contrast group of sanitation brine, each group of the compound production should all be injected 3 mg/kg hydrogen bromic acid scopolamine, put the little whit rats in the Y type labyrinth after ten minutes according to the experiment method in 2.9, record the percentage of wrong reactions, the results are as follows in table 2:

TABLE 2
the influence the compound production acts on the little white rats
which have memory hurt by scopolamine

| | | | | Times of wrong |
|---|---|---|---|---|
| Medicine | Dose (ml/kg) | Taking time | n | reactions ((\overline{X} ± SD)) |
| NS | Same capacity | Bid × 7 | 10 | 0.309 ± 0.158 |
| NS + | 3 mg/kg | Bid × 7 | 10 | 0.538 ± 0.140TT |
| scopolamine | | | | |
| Compound | 4 | Bid × 7 | 10 | 0.370 ± 0.144*** |
| production + | 8 | Bid × 7 | 10 | 0.300 ± 0.110*** |
| scopolamine | | | | |

TTP < 0.01 model group compared to NS
***P < 0.01 compared to model contrast group

3. The Influence the Compound Production Acts on Study Memory of the Elder White

Get elder white rats weighing 18˜20 g and group them by random, ten rats each group. For experiment group fill stomach and give them 4.8 ml/kg compound production, and for blank contrast group give them the same capacity of sanitation brine with the same methods. Give one time a day for seven days. Wait an hour after the last medicine, put the elder rats into a conditioned reflex box with dimention of 35×45×55 cm³, the bottom of the box is a copper bar, on the copper bar is a rubber cushion, the bottom diameter is 5.5 cm with 5.5 cm height.

Let the elder white rat accomodate in the box for five minutes, and put it on the rubber cushion, electrify 25V, record the times the elder white rat could jump on the platform as study index before it stays on the platform for three minutes. Observe the same index after 24 hours as memory index, the results are as follows in table 3:

TABLE 3
the influence the compound production acts on the study memory
of the elder white rats

| | Dose | Taking | | Times of wrong reactions | |
|---|---|---|---|---|---|
| Medicine | (ml/kg) | time | n | (\overline{X} ± SD) | |
| NS | Same | qd × 8 | 10 | 22.0 ± 4.5 | 10.9 ± 4.3 |
| | capacity | | | | |
| Compound | 4 | qd × 8 | 10 | 17.9 ± 3.4** | 6.6 ± 3.0** |
| production | | | | | |
| Compound | 8 | qd × 8 | 10 | 14.5 ± 5.5*** | 4.5 ± 2.0*** |
| production | | | | | |

*P < 0.05
***P < 0.01 contrast to NS

We could draw a conclusion that the compound production not only could improve the study memory of nomal animal, but also could improve the memory hurt impediment by scopolamine, improve study memory and memory hurt.

4. The Influence the Compound Production Acts on SOD of the Elder White Rat

Get elder white male rats and group them by random, ten rats each group. For experiment group fill stomaches with 4.8 ml/kg compound production, and for blank contrast give another group the same capacity of sanitation brine with the same methods. Give one time a day for fifteen days. Wait an hour after the last medicine, take blood from the vein, prepare blood serum with Heparin anticoagulation, determine the activity of blood plusm SOD of the elder white rats, the results are as follows in table 3:

TABLE 4
the influence the compound production acts on SOD
of the elder white rat

| | | | | SOD activity |
|---|---|---|---|---|
| Group | Dose (ml/kg) | Taking time | n | (—X ± SD nu/ml) |
| NS | Same capacity | qd × 15 | 10 | 19.8 ± 3.1 |
| Compound | 4 | qd × 15 | 10 | 24.3 ± 4.1** |
| production | | | | |
| Compound | 8 | qd × 15 | 10 | 27.1 ± 2.9*** |
| production | | | | |

**P < 0.05
***P < 0.01 contrast to NS

5. The Influence the Compound Production Acts on Liver Peroxide Lipid of Elder White Rats

Take the animal used in experiment 4, take out the blood and kill it by disjoint at once, take out the brain and liver rapidly under ice bath condition and make them into 5% sanitation brine equal serum, determine the lipid peroxide in the brain and liver of the elder white rates. The results are as follows in table 5:

TABLE 5
the influence the compound production acts on liver peroxide lipid of elder white rats

| | | | | peroxide lipid (\overline{X} ± SD md/g) | |
|---|---|---|---|---|---|
| | | Taking | | wet layer organization | |
| Group | dose(ml/kg) | time | n | Brain | Liver |
| NS | Same | qd × 7 | 10 | 254.8 ± 13.7 | 190.1 ± 17.9 |
| | capacity | | | | |
| Compound production | 4 | qd × 7 | 10 | 180.8 ± 12.1*** | 143.4 ± 8.6*** |
| Compound production | 8 | qd × 7 | 10 | 163.6 ± 30.1*** | 129.2 ± 20.6*** |

*P < 0.05
***P < 0.01 contrast to NS

We could draw a conclusion from table 4 and table 5 that the compound production could decrease distinctly the form of lipid peroxide in the brain and liver of the elder white rats, improve remarkably the activity of SOD in blood, and present a conection of quantity and effect.

6. The Sedation Function of Compound Production

Get the little white male rats weighing 18˜21 g and group them by random, ten rats each group. For experiment group fill stomach and give them 0.1, 0.2 ml/kg compound production, and for blank contrast group give them the same capacity of sanitation brine with the same methods. After thirty minutes, put the little into GJ-7902 type three-light-path record instrument for the rats' movement, after accommodate for 5 minutes, record the times of the rats' movement, the results are as follows in table 3. We understand from table 6 that the compound production has better sedation function.

TABLE 6
The sedation function of compound production

| | | | times of the rats' |
|---|---|---|---|
| Group | dose (ml/kg) | n | movement (\overline{X} ± SD) |
| NS | — | 10 | 45.6 ± 26.4 |
| Compound | 0.2 | 10 | 8.4 ± 5.6***T |
| production | | | |
| Compound | 0.1 | 10 | 41.7 ± 18.6* |
| production | | | |

*P > 0.05
***P < 0.01 contrast to NS

7. The Hypnosis Function of the Compound Production

Get the little white male rats weighing 18˜22 g and group them by random, 25 rats each group. Fill stomach respectively and give them 0.08, 0.3, 0.4 ml/kg compound production and 30 mg/kg Pentobarbital sodium and for blank contrast give them the same capacity of sanitation brine. Then put the little white rats in infant case under constant temperature of 37° C., observe the sleep time of the little white rats, take the disappearance for continuous 30 seconds of the positive and negative reflect as sleep index, the results are as table 7, we could understand from table 7 thaat the high dose group of 0.4 ml/10 g of compound production could make most animals sleep, but sleep lighter, the group taking 30 mg/kg Pentobarbital sodium could all sleep, this indicates that compound production has sedation function.

TABLE 7
The hypnosis function of the compound production

| | Dose | | Quantity of sleeping animal | | | |
|---|---|---|---|---|---|---|
| Group | (ml/10 g) | n | 5′ | 30′ | 60′ | 90′ |
| Compound | 0.08 | 25 | 0 | 0 | 0 | 0 |
| production | | | | | | |
| Compound | 0.30 | 25 | 0 | 2 | 0 | 0 |
| production | | | | | | |
| Compound | 0.40 | 25 | 23*TT | 25*TT | 15*TT | 0 |
| production | | | | | | |
| Pentobarbital | 30 mg/kg | 25 | 20 TT | 25 TT | 21 TT | 13 |
| sodium NS | same capacity | 25 | 0 | 0 | 0 | 0 |

P > 0.05 contrast to Pentobarbital sodium TT P < 0.01 contrast to NS

### Brief Summary and Discuss

The medicine effect study indicates that the production produced by sixteen kinds of plant compound to improve memory has the effect of sedation and hypnosis, it could improve study memory of elder and little white rats, improve distinctly the memory hurt impediment by scopolamine.

The compound production could decrease distinctly the form of LPO in the brain and liver, improve remarkably the activity of SOD in our body, clear up the freedom radix, protect the cells from suffering the hurt of LPO and resist senescence.

Table 4 the Clinical Observation of the Production Produced by Sixteen Kinds of Plant Compound to Improve Memory

The function decays and malajustment appears when people getting older, especially as they stepping into the middle and old ages, the common symptoms are forgettery, no sleep, heart-throb, more dream, ache in waist and knees, vertigo, tingle and deaf, sex slip etc. All these have affect seriously the living quality and working efficiency of the wrinkly and the elderly, the production produced by sixteen kinds of plant compound to improve memory (in brief compound production), has the pharmic activity of improve memory and treat insomnia. In order to test the clinical curative effect, we have made clinical effective observation to 100 patients, the reports are as follows:

Observation Index:

1. Curative effect observation: observe the clinical symptoms objective characters before and after treatment and the improvemens and changes of electroencephalogram and LPO.

2. Security observation: understand the security of the medicine by observing changes of blood routine, stale routine and liver function before and after treatment.

Treatment Result:

(I) Compare of Clinical Curative Effect Between Two Groups

we can see from table 1 that the percentage of above distinct effect in compound production group and contrast group are 45%, 15% respectively, the total effect percentage are 89%, 73.3% respectively, the P values are <0.01 [symbol] 0.05 respectively by X² test, the two groups are of remarkable differences.

TABLE 1
Compare of clinical effect between two groups

| | | | | | | Percentage of | |
|---|---|---|---|---|---|---|---|
| | | Clinical | Distinct | | | above distinct | Total effect |
| Group | No. | recovery | effect | Effect | No effect | effect | percentage |
| Compound | 100 | 4 | 41 | 44 | 11 | 45% | 29% |
| production | | | | | | | |
| Contrast | 60 | 0 | 9 | 35 | 16 | 15% | 73.3% |

(II) Relation Between Important Symptoms and Period of Treatment and Curative Effect
1. Relation Between Insomnia and Period of Treatment and Curative Effect

we can see from table 2 that the integral value decrease 138 points in compoud production group after taking medicine for two weeks, the decrease rate is 29.9%, decrease 396 points after four weeks, the decrease rate is 85.9%, P<0.01 by X² test, the two groups are of remarkable differences. The integral decrease rate exceeds 2/3 after treating for four weeks. It indicates that the compound production has distinct curative effect to insomnia, The curative effect of taking the compound production for four weeks is distinctly better than that for two weeks.

TABLE 2
Relation between important symptoms and period of treatment and curative effect

| | | | After two weeks | | After four weeks | |
|---|---|---|---|---|---|---|
| | | Total integal | Decrease | Decrease | Decrease | Decrease |
| Group | No | before treatment | points | rate | points | rate |
| Compound production | 98 | 461 | 138 | 29.9% | 396 | 85.9% |
| Contrast group | 69 | 298 | 37 | 12.4% | 201 | 67% |

2. Relation Between Ache in Waist and Knees and Period of Treatment and Curative Effect

we can see from table 3 that the integral value and decrease rate are 64 points, 17.6%; 219 points, 60.2% respectively, P<0.01 by x² test, the two groups are of remarkable differences. It indicates that the curative effect of taking the compound production for four weeks is distinctly better than that for two weeks, and the decrease rate is above one third of total integal value after taking the comoundd production for four weeks, it indicates that the curative effect to this symptom is positive.

TABLE 3
relations between ache in waist and knees and period of treatment and curative effect

| | | | After two weeks | | After four weeks | |
|---|---|---|---|---|---|---|
| | | Total integal | Decrease | Decrease | Decrease | Decrease |
| Group | No | before treatment | points | rate | points | rate |
| Compound production | 91 | 364 | 64 | 17.6% | 219 | 60.2% |
| Contrast group | 52 | 220 | 22 | 10% | 121 | 45% |

3. Relation Between Forgettery and Period of Treatment and Curative Effect

we can see from table 4 that the decrease point and decrease rate of the compound production group after taking for two weeks and four weeks are 52 points, 11% 125 points, 27% respectively, P<0.01 by X² test, the two groups are of remarkable differences. It indicates that the curative effect of taking the compound production for four weeks is distinctly better than that for two weeks.

TABLE 4
relations between forgettery and period of treatment and curative effect

| | | | After two weeks | | After four weeks | |
|---|---|---|---|---|---|---|
| | | Total integal | Decrease | Decrease | Decrease | Decrease |
| Group | No | before treatment | points | rate | points | rate |
| Compound production | 98 | 449 | 52 | 11% | 125 | 27% |
| Contrast group | 60 | 301 | 8 | 2.6% | 177 | 41.2% |

(III) The Changes of Leading Physical and Chemical Indexs and Routine Check Before and After Treatment

The electroencephalograms are nomal before and after treatment in the compound production group, LPO is 18.56±4.43 nmol/ml before treatment, which is above normal level, and decreases to 10.47±4.06 nmol/ml after taking the compound production, which is normal. P<0.01 by T test, there exsists remarkable difference. The compound production could decrease the LPO in the blood distinctly. The blood routine, stale routine and liver function doesn't change after taking the compound production before and after treatment. There doesn't appear any poisonous and side effect in the period of treatment.

### Discuss and Brief Summary

1. The total effective rate and percentage of above distince effect are 89%, 45% respectively by treating 100 patients in clinic with the production produced by sixteen kinds of plant compound to improve memory, and it could improve memory, lighten and improve forgettery.

2. The compound production have distinct effect on main symptoms such as no sleep, ache in waist and knees and on common subsidiary symptoms such as heart-throb, many dreams and deaf and tingle. And also have certain effect on subsidiary symptom as wearness and lack of energy etc.

3. By compareing the curetive effect between two weeks and four weeks of treating three main symptoms such as forgettery, insomnia and ache in waist and knees, we can see that the curetive effect of the compound production of four weeks is better than that of two weeks. This has provided certain clinical basis for the period of treatment of the compound production.

4. The compound production could decrease the LPO in the blood, this indicates that the medicine has certain effect of anti-oxide, anti free radix and anti-senescence.

5. The routine indexes of the compound production such as blood, stale and liver function havn't changed distinctly during observative period of treatment, and there have appeared no poisonous and side effects. The compound production has better curative effect, can be easity taken with good tastes.

### DETAILED DESCRIPTION OF THE INVENTION

1. The pharmic activity and manufacturing method of the production produced by sixteen kinds of plant compound to improve memory is a kind of combination patent of American patents which is combined by the methods of natural production pharmic activity and separation, purification natural production. The production produced by sixteen kinds of plant compound to improve memory is consist of Rhizoma Acori Tatarinowii. Radix Rehmanniae, Radix Ginseng, Radix Polygoni Multiflori, Cortex Eucommiae, Fructus Corni, Fructus Schisandrae, Radix Polygalae, Poria, Semen Plalycladi, Radix Saposhnikovia, Balbus Frtillariae Cirrhosae, Radix Ophiopogoins, Radix Salviae Milfiorrhizae, Radix Notoginseng, Radix Glycyrrhizae

2. Add sixteen kinds of plant materia600 kg, 400 kg, 200 kg water per 100 kg and pick up for 3 hours, 2 hours and 1 hours under 100° C. Unite the filtrate from water. Adsorb with WLD resin through the columns filled with WLD resin. Dewater the compose adsorbing on the WLD resin into ethanol with 65% ethanol under normal temperature. Dry by spraying under 85° C. After detecting ration thumbprint atlas elements with high effective liquid item chromatogram detector, make it into standard production.

3. The study of medical effect indicates that the the production produced by sixteen kinds of plant compound to improve memory has the effect of sedation, hypnosis and improving memory, it could improve the study memory of elder and little whit rats, improve memory hurt impediment by scopolamine distinctly. It could decrease LPO in brain and liver, improve the activity of SOD in body, clear up the free radix, protect cells from hurt by LPO, and obtain the objective of anti-senescence. Through clinical observation, it can treat fegettery, insomnia and ache in waist and knees, and validate that the pharmic activity of the compound production is true and exact. The pharmic activity of the compound production has presented a wide application future for its' medical use.

1. I decare that the pharmic activity and manufacturing method of the production produced by sixteen kinds of plant compound to improve memory (including Rhizoma Acori Tatarinowii 8.58%, Radix Rehmanniae 11.35%, Radix Ginseng 5.67%, Radix Polygoni Multiflori 5.67%, Cortex Eucommiae 5.67%, Fructus Corni 5.67%, Fructus Schisandrae 5.67%, Radix Polygalae 5.67%, Poria 2.83%, Semen Plalycladi 5.67%, Radix Saposhnikovia 5.67%, Balbus Frtillariae Cirrhosae 5.67%, Radix Ophiopogoins 11.35%, Radix Salviae Milfiorrhizae 5.67%, Radix Notoginseng 3.52%, Radix Glycyrrhizae 5.67%) to be my invention.
I consider that my invention include that pick up water from the sixteen kinds of plant material, absorb and dewater with WLD resin and dry. After detecting ration thumbprint atlas elements with high effective liquid item chromatogram detector, make it into standard production
I consider that the production produced by sixteen kinds of plant compound to improve memory in my invention could improve the study memory of elder and little white rats, improve memory hurt impediment, decrease the form of LPO in liver and brain, improve the pharmic activity of SOD in sedation, hypnosis and memory. The curative result validates the veracity of the pharmic activity to the patients on forgettery and insomnia in clinic.
